(19) United States
(12) Patent Application Publication
(10) Pub. No.: US 2018/0103890 A1
(43) Pub. Date: Apr. 19, 2018

(54) SYSTEM AND METHOD FOR IMAGING MACROPHAGE ACTIVITY USING DELTA RELAXATION ENHANCED MAGNETIC RESONANCE IMAGING

(71) Applicants: Cameron Anthony PIRON, Toronto (CA); Chad Tyler HARRIS, Toronto (CA); Jeff STAINSBY, Toronto (CA); Alexander Gyles PANTHER, Toronto (CA)

(72) Inventors: Cameron Anthony PIRON, Toronto (CA); Chad Tyler HARRIS, Toronto (CA); Jeff STAINSBY, Toronto (CA); Alexander Gyles PANTHER, Toronto (CA)

(21) Appl. No.: 15/534,037

(22) PCT Filed: Mar. 11, 2015
(86) PCT No.: PCT/IB2015/051762

§ 371 Date: Jun. 8, 2017

(51) Int. Cl. A61B 5/00; A61B 5/055; A61K 49/10; G01R 33/44; G01R 33/56; G01R 33/50; G01R 33/48

(52) U.S. Cl. CPC A61B 5/414; A61B 5/055; A61K 49/101; G01R 33/4806; G01R 33/5601; G01R 33/50; G01R 33/445

(57) ABSTRACT

A magnetic resonance imaging (MRI) system is provided for imaging immune response of soft tissue to therapy by, prior to therapy, administering a contrast agent to the soft tissue; imaging a region of interest using delta relaxation enhanced magnetic resonance (DREMR) to define a functional section; selectively sampling local cells in the functional section; conducting immuno-assay analysis on the sampled local cells; and following therapy, further imaging said region of interest using DREMR to assess immune response of said cells to therapy.

### FIELD

This specification relates generally to magnetic resonance imaging, and specifically to a system and method for producing image contrasts in magnetic resonance imaging.

### BACKGROUND

In the field of medicine, imaging and image guidance are a significant component of clinical care. From diagnosis and monitoring of disease, to planning of the surgical approach, to guidance during procedures and follow-up after the procedure is complete, imaging and image guidance provides effective and multifaceted treatment approaches, for a variety of procedures, including surgery and radiation therapy. Targeted stem cell delivery, adaptive chemotherapy regimes, and radiation therapy are only a few examples of procedures utilizing imaging guidance in the medical field.

Advanced imaging modalities such as Magnetic Resonance Imaging (“MRI”) have led to improved rates and accuracy of detection, diagnosis and staging in several fields of medicine including neurology, where imaging of diseases such as brain cancer, stroke, Intra-Cerebral Hemorrhage (“ICH”), and neurodegenerative diseases, such as Parkinson's and Alzheimer's, are performed. As an imaging modality, MRI enables three-dimensional visualization of tissue with high contrast in soft tissue without the use of ionizing radiation. This modality is often used in conjunction with other modalities such as Ultrasound (“US”), Positron Emission Tomography (“PET”) and Computed X-ray Tomography (“CT”), by examining the same tissue using the different physical principals available with each modality. CT is often used to visualize boney structures, and blood vessels when used in conjunction with an intra-venous agent such as an iodinated contrast agent. MRI may also be performed using a similar contrast agent, such as an intra-venous gadolinium based contrast agent which has pharmaco-kinetic properties that enable visualization of tumors, and break-down of the blood brain barrier. These multi-modality solutions can provide varying degrees of contrast between different tissue types, tissue function, and disease states. Imaging modalities can be used in isolation, or in combination to better differentiate and diagnose disease.

In neurosurgery, for example, brain tumors are typically excised through an open craniotomy approach guided by imaging. The data collected in these solutions typically consists of CT scans with an associated contrast agent, such as iodinated contrast agent, as well as MRI scans with an associated contrast agent, such as gadolinium contrast agent. Also, optical imaging is often used in the form of a microscope to differentiate the boundaries of the tumor from healthy tissue, known as the peripheral zone. Tracking of instruments relative to the patient and the associated imaging data is also often achieved by way of external hardware systems such as mechanical arms, or radiofrequency or optical tracking devices. As a set, these devices are commonly referred to as surgical navigation systems.

The link between immunological response imaging and therapy is critical to managing treatment in a number of areas, such as oncology, MS lesions, stroke penumbra, traumatic brain injury, etc. It is therefore desirable to observe the natural immune response to a tumor or trauma, as well as the immune response being mediated by therapy, for example increased or decreased immune response as a result of tumor or brain injury therapy. Macrophages play a key role in the immunological response; therefore, the ability to image and track macrophage activity in vivo would provide great insight into the immunological response of the body.

Nuclear Magnetic Resonance (NMR) imaging, or Magnetic Resonance Imaging (MRI) as it is commonly known, is a non-invasive imaging modality that can produce high resolution, high contrast images of the interior of a subject. MRI involves the interrogation of the nuclear magnetic moments of a sample placed in a strong magnetic field with radio frequency (RF) magnetic fields. During MRI the subject, typically a human patient, is placed into the bore of an MRI machine and is subjected to a uniform static polarizing magnetic field B0 produced by a polarizing magnet housed within the MRI machine. Radio frequency (RF) pulses, generated by RF coils housed within the MRI machine in accordance with a particular localization method, are typically used to scan target tissue of the patient. MRI signals are radiated by excited nuclei in the target tissue in the intervals between consecutive RF pulses and are sensed by the RF coils. During MRI signal sensing, gradient magnetic fields are switched rapidly to alter the uniform magnetic field at localized areas thereby allowing spatial localization of MRI signals radiated by selected slices of the target tissue. The sensed MRI signals are in turn digitized and processed to reconstruct images of the target tissue slices using one of many known techniques.

When a substance, such as human tissue is subjected to the static polarizing magnetic field B0, the individual magnetic moments of the spins in the tissue attempt to align with the static polarizing magnetic field B0, but precess about the static polarizing magnetic field B0 in random order at their characteristic Larmor frequency. The net magnetization vector lies along the direction of the static polarizing magnetic field B0 and is referred to as the equilibrium magnetization M0. In this configuration, the Z component of the magnetization or longitudinal magnetization MZ is equal to the equilibrium magnetization M0. If the target tissue is subjected to an excitation magnetic field B1, which is in the x-y plane and which is near the Larmor frequency, the longitudinal magnetization MZ may be rotated, or “tipped” into the x-y plane to produce a net transverse magnetic moment MXY. When the excitation magnetic field B1 is terminated, relaxation of the excited spins occurs, with a signal being emitted that effects the magnitude of radiated MRI signals. The emitted signal is received and processed to form an image.

In particular, when the excitation magnetic field B1 is terminated, the longitudinal magnetization MZ relaxes back to its equilibrium. The time constant that describes how the longitudinal magnetization MZ returns to its equilibrium value is commonly referred to as the spin lattice relaxation time T1. The spin lattice relaxation time T1 characterizes the time required to reduce the difference between the longitudinal magnetization MZ and its equilibrium value M0 to zero.

The net transverse magnetic moment MXY also relaxes back to its equilibrium when the excitation magnetic field B1 is terminated. The time constant that describes how the transverse magnetic moment MXY returns to its equilibrium value is commonly referred to as the transverse relaxation time or spin-spin relaxation time T2. The transverse relaxation time T2 characterizes the time required to reduce the transverse magnetic moment MXY to zero. Both the spin lattice relaxation time T1 and the transverse relaxation time T2 are tissue specific and vary with concentration of different chemical substances in the tissue as well as with different microstructural features of the tissue. Variations of the spin lattice relaxation time T1 and/or the transverse relaxation time T2 from normal can also be indicative of disease or injury.

Like many diagnostic imaging modalities, MRI can be used to differentiate tissue types, e.g. muscles from tendons, white matter from gray matter, and healthy tissue from pathologic tissue. There exist many different MRI techniques, the utility of each depending on the particular tissue under examination. Some techniques examine the rate of tissue magnetization, while other techniques measure the amount of bound water or the velocity of blood flow. Often, several MRI techniques are used together to improve tissue identification. In general, the greater the number of tests available the better chance of producing a correct diagnosis.

In some instances contrast agents may be used to emphasize certain anatomical regions. For example, a Gadolinium chelate injected into a blood vessel will produce enhancement of the vascular system, or the presence and distribution of leaky blood vessels. Iron-loaded stem cells injected into the body and detected by MRI, will allow stem cell migration and implantation in-vivo to be tracked. For a contrast agent to be effective the contrast agent must preferentially highlight one tissue type or organ over another. Furthermore, the preferential augmentation of signal must be specific to the particular tissue type or cell of interest.

All contrast agents will shorten the T1 and T2 relaxation times of nearby tissue; however, it is useful to subdivide them into two main groups. T1 contrast agents, or “positive” agents, decrease T1 approximately the same amount as T2, these agents typically give rise to increases in signal intensity in images. Examples of T1 agents are paramagnetic gadolinium- and manganese-based agents. The second group can be classified as T2 contrast agents, or “negative” agents, these agents decrease T2 much more than T1 and hence typically result in a reduction of signal intensity in images. Examples of T2 contrast agents are ferromagnetic and superparamagnetic iron oxide based particles, commonly referred to as superparamagnetic iron oxide (SPIO) and ultra-small superparamegnetic iron oxide (USPIO) particles.

Contrast agents can further be classified as targeted or non-targeted. A targeted contrast agent has the ability to bind to specific molecules of interest. In some cases, the T1 relaxation time of the agent significantly decreases upon binding. For example, MS-325 is an agent that binds to serum albumin in the blood. For many agents (including MS-325), the T1 relaxation time of the agent in the bound state is a strong function of the magnetic field strength. When this is the case (i.e. a molecule's T1 relaxation time is a strong function of the magnetic field strength), the molecule is said to have T1 dispersion.

Delta relaxation enhanced magnetic resonance (DREMR), generally referred to as field-cycled relaxometry or field-cycled imaging is an MRI technique that relies on using underlying tissue contrast mechanisms that vary with the strength of the applied magnetic field in order to generate novel image contrasts. To achieve DREMR contrast, the main magnetic field is varied as a function of time during specific portions of an MR pulse sequence. A field-shifting electromagnet coil is used to perform the field variation. The DREMR method exploits the difference in the T1 dispersion property (variation of T1 with field strength) of targeted T1 contrast agents in the bound and unbound states in order to obtain an image that contains signal only from contrast agent that is in the bound state, while suppressing signal from contrast agent in the unbound state.

It is well known, however not yet exploited, that the T1 relaxation time of iron oxide based contrast agents also varies with the strength of the magnetic field. Therefore, the DREMR method can be used in order to obtain images that contain signal specifically where the iron oxide based contrast agents have accumulated.

Relatively recently, iron oxide nanoparticles have become the preferred approach to track macrophage activity within the body. This is achievable because macrophages have naturally high endocytosis activity and hence will “eat” the contrast agent after it has been injected into the subject. Once a substantial amount of contrast agent has accumulated in the macrophage and/or a substantial amount of macrophages containing minute amounts of contrast agent have accumulated, the signal will decrease in the immediate area due to the shortening of T2 caused by the contrast agent. This change in signal can be detected by use of subtraction between pre- and post-injection images.

There are a few problems with the above approach, the first is the dependence on a subtraction between pre- and post-injection images. These images must be taken at different times and tissue may move between scans causing subtraction artifacts. One may wish to avoid this dependence of a pre-injection scan simply by monitoring locations where there is signal dropout, this brings up the second issue with the above approach: signal dropout can be caused by other, non-contrast related, phenomena; for example, susceptibility differences between tissues. If there is already signal dropout present due to other phenomena, additional signal dropout cannot be detected. The previous problem described, not being able to detect additional signal dropout if it is already present, points to a third problem with the aforementioned technique to monitor macrophage activity: once enough contrast agent has accumulated to produce adequate signal dropout, additional accumulation cannot be detected. This leads to a maximum concentration of contrast agent that can be detected within a certain region, thereby making the above mentioned method to track macrophage activity non-quantifiable.

### SUMMARY

It is an object to provide a novel system and method for observing immune response or macrophage activity as seen with SPIO/USPIO uptake into macrophages through DREMR mediated contrast by exploiting the T1 dispersion property of iron oxide based contrast agents that obviates and mitigates at least one of the above-identified disadvantages of the prior art.

By using a field-shifting MR system it is possible to selectively obtain contrast from tissue that has T1 dispersion (i.e. tissue T1 relaxation time that strongly depends on the main magnetic field strength). This is can be achieved by modulating the polarizing magnetic field of the system during the longitudinal magnetization relaxation recovery portion of the MR pulse sequence, obtaining two images or data sets at two distinct polarizing field-strengths, and then processing said images or data sets in order to extract information related to the aforementioned T1 dispersion property.

In accordance with one aspect, there is provided a diagnostic method for imaging immune response of soft tissue to therapy using a magnetic resonance imaging system, wherein the method comprises, prior to therapy: administering a contrast agent to the soft tissue; imaging a region of interest using delta relaxation enhanced magnetic resonance (DREMR) to define a functional section; selectively sampling local cells in the functional section; conducting immuno-assay analysis on the sampled local cells; and following therapy: further imaging said region of interest using DREMR to assess immune response of said cells to therapy.

In accordance with another aspect, there is provided a delta relaxation magnetic resonance imaging (DREMR) system for imaging immune response of soft tissue to therapy according to the method set forth in the previous paragraph, comprising: a main field magnet generating a main magnetic field at an imaging volume; and an integrated magnet device placed within the bore of the main magnet, the integrated magnet device comprising field-shifting electromagnets; gradient coils; and at least one substrate layer providing mechanical support for the field-shifting electromagnets and the gradient coils.

According to the system and method of the present invention, where the DREMR method is used to selectively image where nanoparticles, such as SPIOs or USPIOs, are located within tissue, as set forth in the previous two paragraphs, a number of applications are possible, for example: locating reactive brain cells (e.g. astrocytes and macrophages) in or at the margins of brain tumors; intra-operative surgical resection assessment; and screening for tumor metastases.

These, together with other aspects and advantages which will be subsequently apparent, reside in the details of operation as more fully hereinafter described and claimed, reference being had to the accompanying drawings forming a part hereof, wherein like numerals refer to like parts throughout.

### BRIEF DESCRIPTION OF THE DRAWINGS

Embodiments will now be described, by way of example only, with reference to the drawings, in which:

FIG. 1 shows a block diagram of functional subsystems of a delta relaxation enhanced magnetic resonance (DREMR) imaging system in accordance with an implementation.

FIG. 2A shows an example DREMR pulse sequence utilizing a “positive” (enhancing) polarizing field-shift.

FIG. 2B shows an example DREMR pulse sequence utilizing a “negative” (decreasing) polarizing field-shift.

FIG. 3 shows an example “positive” field-shift image, “negative” field-shift image, subsequent subtracted image (positive field-shift image minus negative field-shift image), intensity correction image, and the final normalized subtracted image.

FIG. 4 is a flowchart showing steps for using the DREMR imaging method of FIGS. 1-3 to visualize macrophage activity and response to therapy after administration of iron oxide based contrast agents.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

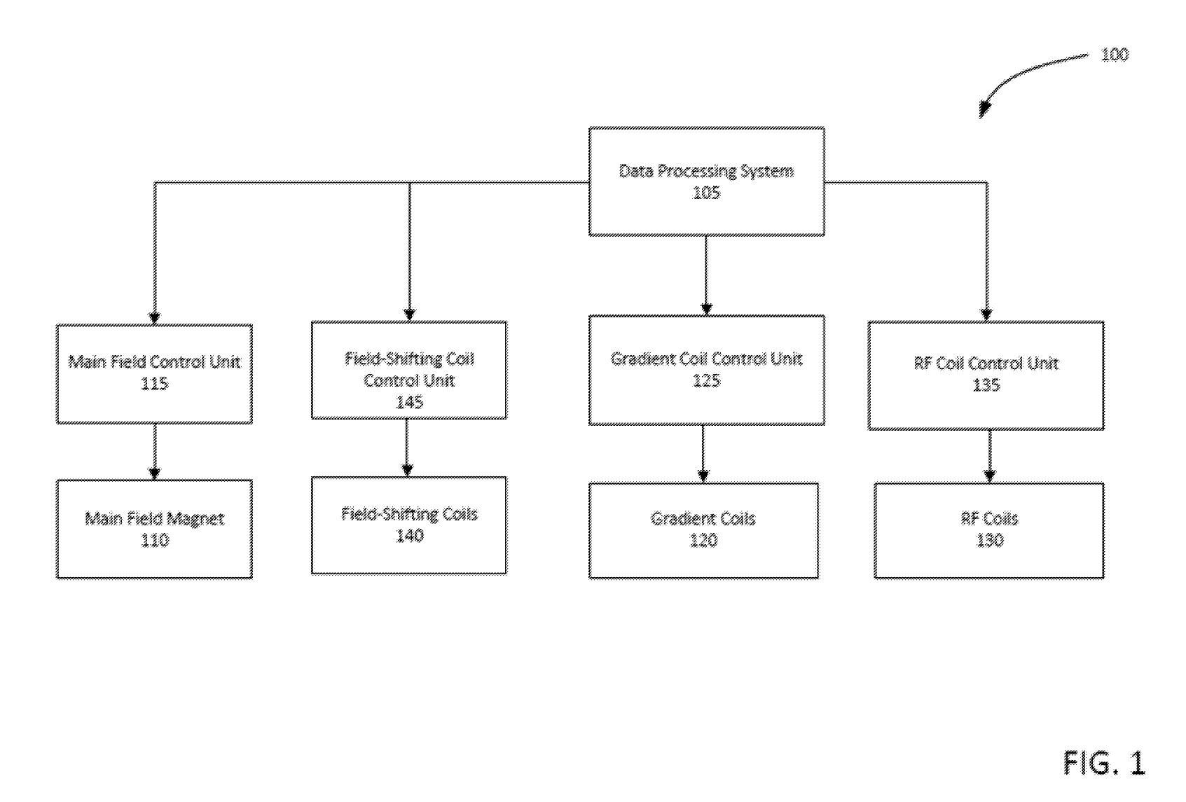

Referring to FIG. 1, a block diagram of a delta relaxation magnetic resonance imaging (DREMR) system, in accordance with an example implementation, is shown at 100. The example implementation of the DREMR system indicated at 100 is for illustrative purposes only, and variations including additional, fewer and/or varied components are possible. Traditional magnetic resonance imaging (MRI) systems represent an imaging modality which is primarily used to construct pictures of nuclear magnetic resonance (MR) signals from protons such as hydrogen atoms in an object. In medical MRI, typical signals of interest are MR signals from water and fat, the major hydrogen containing components of tissues. DREMR systems use field-shifting magnetic resonance methods in conjunction with traditional MRI techniques to obtain images with different contrast than is possible with traditional MRI, including molecularly-specific contrast.

As shown in FIG. 1, the illustrative DREMR system 100 comprises a data processing system 105. The data processing system 105 can generally include one or more output devices such as a display, one or more input devices such as a keyboard and a mouse as well as one or more processors connected to a memory having volatile and persistent components. The data processing system 105 can further comprise one or more interfaces adapted for communication and data exchange with the hardware components of MRI system 100 used for performing a scan.

Continuing with FIG. 1, the exemplary DREMR system 100 can also include a main field magnet 110. The main field magnet 110 can be implemented as a permanent, superconducting or a resistive magnet, for example. Other magnet types, including hybrid magnets suitable for use in the DREMR system 100 will be known to a person of skill and are contemplated. The main field magnet 110 is operable to produce a substantially uniform main magnetic field having strength B0 and a direction along an axis. The main magnetic field is used to create an imaging volume within which desired atomic nuclei of an object, such as the protons in hydrogen within water and fat, are magnetically aligned in preparation for a scan. In some implementations, as in this example implementation, a main field control unit 115 can communicate with data processing system 105 for controlling operation of the main field magnet 110.

The DREMR system 100 can further include gradient magnets, for example gradient coils 120 used to produce deliberate variations in the main magnetic field (B0) along, for example, three perpendicular gradient axes. The size and configuration of the gradient coils 120 can be such that they produce a controlled and uniform linear gradient. For example, three paired orthogonal current-carrying coils located within the main field magnet 110 can be designed to produce desired linear-gradient magnetic fields. The variation in the magnetic field permits localization of image slices as well as phase encoding and frequency encoding spatial information.

The magnetic fields produced by the gradient coils 120, in combination and/or sequentially, can be superimposed on the main magnetic field such that selective spatial excitation of objects within the imaging volume can occur. In addition to allowing spatial excitation, the gradient coils 120 can attach spatially specific frequency and phase information to the atomic nuclei placed within the imaging volume, allowing the resultant MR signal to be reconstructed into a useful image. A gradient coil control unit 125 in communication with the data processing system 105 can be used to control the operation of the gradient coils 120.

The DREMR system 100 can further comprise radio frequency (RF) coils 130. The RF coils 130 are used to establish an RF magnetic field with strength B1 to excite the atomic nuclei or “spins” within an object being imaged. The RF coils 130 can also detect signals emitted from the “relaxing” spins within the object. Accordingly, the RF coils 130 can be in the form of separate transmit and receive coils or a combined transmit and receive coil with a switching mechanism for switching between transmit and receive modes.

The RF coils 130 can be implemented as surface coils, which are typically receive-only coils and/or volume coils which can be receive-and-transmit coils. The RF coils 130 can be integrated in the main field magnet 110 bore. Alternatively, the RF coils 130 can be implemented in closer proximity to the object being imaged, such as a head, and can take a shape that approximates the shape of the object, such as a close-fitting helmet. An RF coil control unit 135 can be used to communicate with the data processing system 100 to control the operation of the RF coils 130.

In order to create a contrast image in accordance with field-shifting techniques, DREMR system 100 can use field-shifting electromagnets 140 while generating and obtaining MR signals. The field-shifting electromagnets 140 can modulate the strength of the main magnetic field. Accordingly, the field-shifting electromagnets 140 can act as auxiliary to the main field magnet 110 by producing a field-shifting magnetic field that augments or perturbs the main magnetic field. A field-shifting electromagnet control unit 145 in communication with the data processing system 100 can be used to control the operation of the field-shifting electromagnets 140.

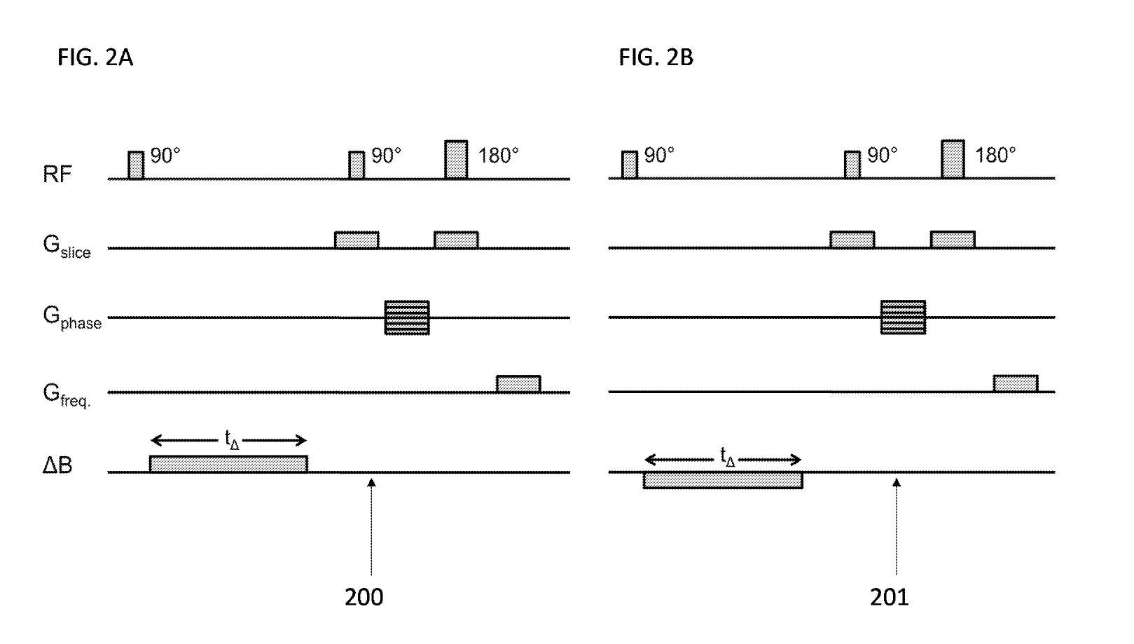

There are many techniques for obtaining images that will produce contrast related to the T1 dispersion of tissue using the DREMR system 100. To provide an illustration of this, simplified operations for obtaining an image with contrast specific to the change in relaxation rate (1/T1) between two distinct polarizing magnetic field strengths will be described as a non-limiting example. Referring now to FIG. 2A and FIG. 2B, illustrative DREMR pulse sequences are shown. Specifically, timing diagrams for the example pulse sequences are indicated. The timing diagrams show pulse or signal magnitudes, as a function of time, for transmitted (RF) signal, magnetic field gradients (Gslice, Gphase, and Gfreq), and field-shifting signal (AB). The RF pulses can be generated by the transmit aspect of the RF coils 130. The waveforms for the three gradients can be generated by the gradient coils 120. The waveform for the field-shifting signal can be generated by the field-shifting electromagnet 140. The precise timing, amplitude, shape, and duration of the pulses or signals may vary for different imaging techniques. For example, the field-shifting signal may be applied for a shorter or longer duration or at a larger or smaller amplitude such that the image contrast due to T1 dispersion is optimized.

Referring now to FIG. 2A, the first event to occur in pulse sequence 200 can be to apply an RF pulse such that it produces a 90 degree rotation of the magnetization from the z-axis (the direction of the main magnetic field) into the xy-plane (the plane of detection of the receiver coils). This has the effect of making the magnetization along the z-axis, denoted Mz, zero. Once the first 90 degree RF pulse has finished, the field-shifting electromagnet can be turned on for a time period of t_{Δ}, in this first sequence the field-shifting electromagnet is turned on such that the field that is produced is additive to (i.e. increases) the main magnetic field. Once the field-shifting electromagnet is turned off the pulse sequence can continue with a particular imaging sequence. In this example implementation, the imaging sequence that is used is a spin-echo sequence.

Referring now to FIG. 2B, once again the first event to occur in pulse sequence 201 can be to apply an RF pulse such that it produces a 90 degree rotation of the magnetization from the z-axis (the direction of the main magnetic field) into the xy-plane (the plane of detection of the receiver coils). This has the effect of making the magnetization along the z-axis, denoted Mz, zero. Once this first 90 degree RF pulse has finished, the field-shifting electromagnet can be turned on for a time period of t_{Δ}, in this second sequence the field-shifting electromagnet is turned on such that the field that is produced is subtracted from (i.e. decreases) the main magnetic field. Once the field-shifting electromagnet is turned off the pulse sequence can continue with a particular imaging sequence. In this example implementation, the imaging sequence that is used is a spin-echo sequence.

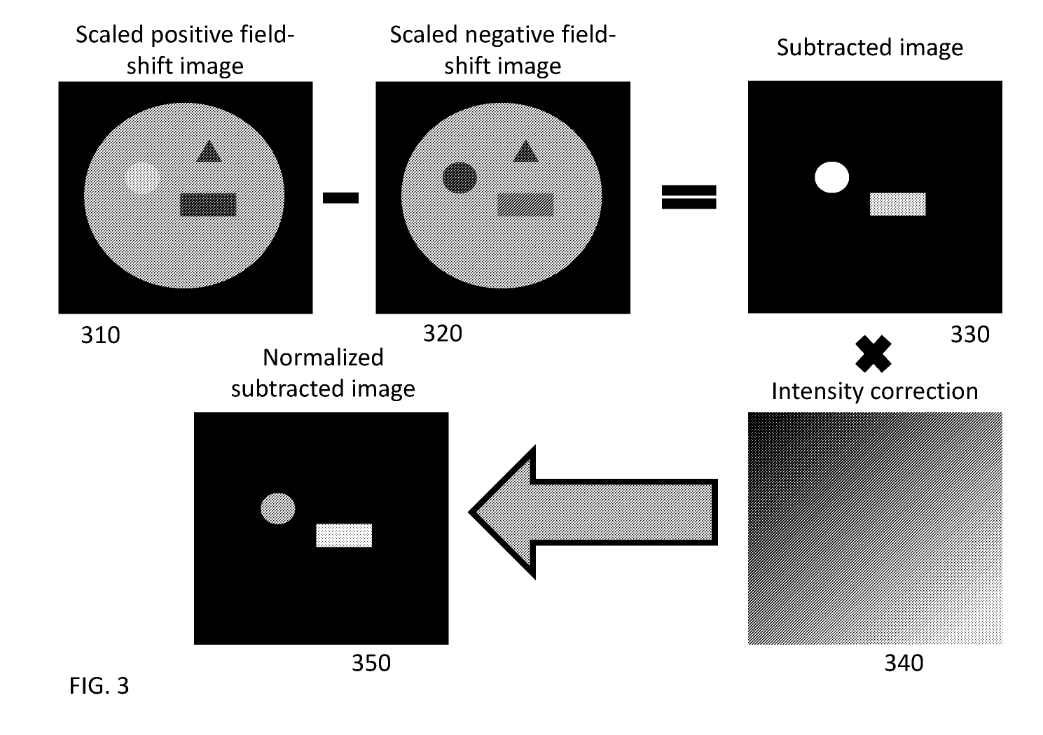

Referring now to FIG. 3, there is an image corresponding to the positive field-shift sequence from FIG. 2A denoted “scaled positive field-shift image” at 310, the word “scaled” has been added to the description of this image to indicate the multiplication by a scalar factor needed prior to subtraction (see DREMR reference). Similarly, there is an image corresponding to the negative field-shift sequence from FIG. 2B denoted “scaled negative field-shift image” at 320, once again the word “scaled” has been added to the description to indicate the multiplication by a scalar factor that is needed prior to subtraction. These two images can be subtracted from each other to produce a “subtracted image” as indicated at 330. Due to inhomogeneities in the polarizing field that is produced by the field-shifting electromagnet (i.e. the field-shift in one region of space may be slightly larger than the field-shift in another region of space), the subtracted image must be multiplied by an intensity correction image (340) on a pixel-by-pixel basis. The intensity correction image 340 can be calculated as the inverse of 1 plus the difference between the field-shift at each pixel location from the field-shift at iso-center (the center of the imaging region), divided by the field-shift at isocenter. After multiplying the subtracted image 330 by the intensity correction image 340 the result is the “Normalized subtracted image” at 350. It is important to note that the field-shift images do not necessarily need to be “positive” (i.e. adding to the main field) and “negative” (i.e. subtracting from the main field), they must only be at two distinct polarizing fields.

According to the present invention, MRI contrast agents, such as SPIOs and USPIOs are injected into tissue. The contrast agent is subsequently engulfed by inflammatory cells (macrophages), with the result that MRI signal due to T1 dispersion (i.e. signal produced using the DREMR methodology described above) correlates with macrophage density.

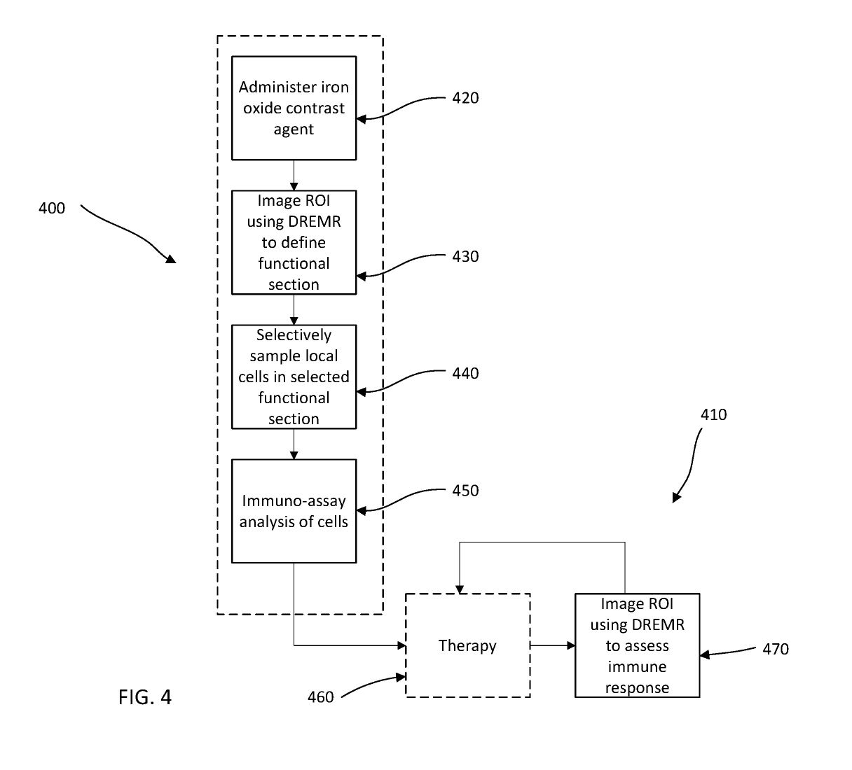

According to one aspect of the present invention, the DREMR imaging system of FIGS. 1-3 may be used to visualize immune response by administering iron oxide based contrast agents, according to the steps set forth in FIG. 4, wherein part 400 shows steps for visualizing the natural immune response of tissue in a region of interest (ROI), and part 410 shows steps of visualizing the immune response being mediated by therapy (e.g. increased immune response resulting from immunologically responsive tumor therapy, or decreased immune response due to brain (or other) injury therapy.

At 420, a contrast agent is administered (e.g. via injection). In one embodiment, the contrast agent is a nanoparticle, such as superparamagnetic iron oxide (SPIO) or ultra-small superparamagnetic iron oxide (USPIO). At 430, the ROI is imaged using DREMR imaging, to define a functional section (e.g. of a tumor or trauma to be treated). In this example implementation, the term “functional section” is defined as a region of interest where signal produced by the DREMR methodology is larger than a pre-defined threshold. It is important to note that the criteria for a functional section may change for other implementations, such as being larger than a given threshold and also being located in the immediate vicinity of a known region of trauma, and is contemplated.

Selective Analysis is then perfomed on a functional section, at steps 440 and 450. In one embodiment, at 440, local cells within the functional section are selectively sampled (e.g. via biopsy) and then, at 450, immuno-assay analysis is conducted on the sampled cells in the selected area (e.g. to identify the natural targets of the tumor). In alternate embodiments, selective analysis performs comparison of cells within region of interest of known types to a database or informatics system.

Then, at 460, appropriate therapy is performed based on the diagnostic process of part 400. At 470, the ROI is again imaged using DREMR imaging to assess immune response and adjust therapy 460 for enhancing the immuno-response to these cells. Note that the actual therapy 460 does not form part of the diagnostic method of the present invention.

The absolute signal in the DREMR subtraction image at 430 and 470 depends on the contrast agent concentration which, assuming sufficient uptake, is dependent on the level of macrophage activity. Thus, the amount of signal in the DREMR subtraction image is correlated with the absolute level of macrophage activity. Therefore, according to the present invention, the amount of signal in the DREMR subtraction image may be used to measure the response of tissue to therapy where the application of therapy is aimed to have a specific increase or decrease in the immune-response in tissue, as quantified by the DREMR subtraction images taken at different time points during therapy (i.e. initially at 430 and successively and repeatedly at 470).

According to further aspects of the invention, several applications of the system and method set forth above are contemplated.

In one application, DREMR imaging is performed at 430 to locate reactive brain cells (e.g. astrocytes and macrophages) in or at the margins of brain tumors and in locations not otherwise identified by MR imaging methods. Using the location of reactive brain cells identified in this manner therapy 460 may be specifically targeted (e.g. to guide margins of tumor resection, guide injection of immuno-response specific therapeutic agents, guide tissue biopsy, etc.)

In a surgical application, since SPIOs have been demonstrated to accumulate in areas of active macrophages over the course of many hours and remain detectable for 2-5 days post injection, DREMR imaging may be performed intra-operatively at 470 to assess the extent of surgical resection. Other intra-operative MR imaging methods which rely on tissue contrast mechanisms may become intra-operatively compromised (e.g. T2-mediated contrast that can be confounded by bleeding or fluid accumulation in the resection cavity; Gd contrast-enhanced imaging which can be confounded by Gd leaking into the resection cavity; and other acute vascular permeability changes due to the surgical process, not related to tumor vascularity). According to an aspect of the invention, intra-operative DREMR imaging at 470 may be used to detect SPIOs that have been administered pre-operatively at 420, to visualize residual reactive tissue targets for further resection.

In another diagnostic application, DREMR imaging in accordance with 400 and 410 may be used to screen for tumor metastases (e.g. by locating SPIOs that have accumulated in areas of active tumors).

Although the applications set forth in detail above are directed at managing immune response in neurological treatment such as treating brain tumors and injuries, the DREMR imaging with SIPO contrast enhancement as set forth herein may be applied to all areas of oncology as well as the identification and treatment of MS lesions, stroke penumbra, etc.

The specific embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

Therefore what is claimed is:

1. A diagnostic method for imaging immune response of soft tissue to therapy using a magnetic resonance imaging system, comprising:
prior to therapy, administering a contrast agent to the soft tissue;
imaging a region of interest using delta relaxation enhanced magnetic resonance (DREMR) to define a functional section;
perform selective analysis on the functional section; and
following therapy, further imaging said region of interest using DREMR to assess immune response of said cells to therapy.

2. The method of claim 1, wherein said contrast agent is a nanoparticle.

3. The method of claim 2, wherein said nanoparticle is superparamagnetic iron oxide (SPIO).

4. The method of claim 2, wherein said nanoparticle is ultra-small superparamagnetic iron oxide (USPIO).

5. The method of claim 1, wherein said contrast agent is administered via injection.

6. The method of claim 1, wherein imaging said region of interest using DREMR further comprises modulating a polarizing field of the magnetic resonance imaging system during the longitudinal relaxation recovery portion of an MR pulse sequence, for obtaining two images at two distinct polarizing fields, scaling said images, subtracting one said image from the other said image, then finally performing intensity correction on said subtracted image to generate a normalized subtraction image.

7. The method of claim 1, wherein said selective analysis comprises selectively sampling local cells in the functional section and conducting immuno-assay analysis on the sampled local cells.

8. The method of claim 1, wherein said selective analysis comprises comparison of cells within region of interest to a database of known type.

9. The method of claim 7, wherein said selectively sampling comprises biopsy.

10. The method of claim 1, wherein said immune response comprises one of either an increase resulting from immunologically responsive tumor therapy, or a decrease responsive to therapeutically effective injury therapy.

11. The method of claim 6, wherein immune response of said cells to therapy is represented by the magnitude of said normalized subtraction image which is dependent on amount of contrast agent uptake in said cells which is dependent on level of macrophage activity.

12. The method of claim 1, wherein said further imaging is conducted at different times during said therapy.

13. The diagnostic use of the method according to claim 1 to locate reactive brain cells in or at the margins of brain tumors, for targeting said therapy.

14. The diagnostic use of the method according to claim 1 to assess extent of surgical resection.

15. The diagnostic use according to claim 13 to further detect contrast agent that has been administered pre-operatively via said further imaging for visualizing residual reactive tissue targets for further resection.

16. Diagnostic use of the method according to claim 1 to screen for tumor metastases by locating contrast agent that has accumulated in areas of active tumors.

17. A delta relaxation magnetic resonance imaging (DREMR) system for imaging immune response of soft tissue to therapy according to claim 1, comprising:
a main field magnet generating a main magnetic field at an imaging volume; and
an integrated magnet device placed within the bore of the main magnet, the integrated magnet device comprising:
field-shifting electromagnets;
gradient coils; and
at least one substrate layer providing mechanical support for the field-shifting electromagnets and the gradient coils.
